(19) United States
(12) Patent Application Publication
(10) Pub. No.: US 2003/0211988 A1
(43) Pub. Date: Nov. 13, 2003

(54) Enhancing lymph channel development and treatment of lymphatic obstructive disease

(76) Inventor: Stephen E Epstein, Rockville, MD (US)

Correspondence Address: Vorys Sater Seymour and Pease, Suite 1111, 1828 L Street N W, Washington, DC, 20036-5104, US

(21) Appl. No.: 10/169,838

(22) PCT Filed: Jan. 9, 2001
(86) PCT No.: PCT/US01/00545

(51) Int. Cl. A61K 38/18

(52) U.S. Cl. 514/12

(57) ABSTRACT

Disclosed and claimed are compositions and methods for therapy and/or prevention of lymphedema. The compositions can include an agent that induces development of lymphatic channels or lymphangiogenesis, such as, VEGF-C and/or that which stimulates VEGF-C expression and/or that which stimulates VEGF-C expression or that which stimulates its interaction or that which stimulates other pathways to so stimulate the development of lymphatic channels or lymphangiogenesis or that which stimulates along any point of or any molecules involved in the signal transduction pathway leading to lymphangiogenesis or lymph channel development (and/or vector(s) expressing one or more of these agent(s)). Embodiments can include kits.

### RELATIONSHIP TO OTHER APPLICATIONS

This application claims the benefit of copending U.S. Provisional Application No. 60/175,393, filed Jan. 11, 2000.

### FIELD OF THE INVENTION

This invention relates to the use of a protein or of proteins or a vector or vectors or a combination thereof that can enhance the development of lymphatic channels; for instance, in individuals with obstructed lymph channels (lymphedema). The process by which the enhancement of the development of lymphatic channels occurs is herein termed lymphangiogenesis. The protein(s) can either be injected into the affected site directly as protein(s), or as a vector containing the gene encoding the relevant protein or proteins. The protein(s) can be delivered either directly, or in a vehicle (such as liposomes) that can facilitate delivery of the protein to the targets site. The vector containing the gene can be a naked plasmid DNA vector, or in any other suitable vector that contains the gene. The resulting new or enlarged lymphatic channels enhance lymph conduction around the obstructed channels, thereby alleviating the lymphedema of the tissue drained by the obstructed lymphatics.

The present invention relates to compositions and methods for the preventing and/or treatment of lymphedema.

The present invention further relates to compositions and methods for producing a lymphangiogenic effect and/or for developing or enhancing the development of lymphatic channels, especially in individuals with obstructed lymph channels.

Lymphedema can be caused be various therapies or can occur spontaneously; and thus, the present invention relates to compositions and methods for inhibiting or preventing or controlling lymphedema; for instance, by producing a lymphangiogenic effect and/or for developing or enhancing the development of lymphatic channels, especially in individuals with obstructed lymph channels.

The present invention further relates to compositions and methods containing or employing agents having lymphangiogenic effects, such as VEGF-C or other agents which enhance the development of lymphatic channels. The agent can be a protein or a gene; for instance a gene which expresses a protein in vivo; the gene could be delivered by a vector, e.g., plasmid or viral vector.

The present invention also relates to methods and compositions for administering an agent which induces lymphangiogenesis, and which thereby may inhibit lymphedema.

The present invention yet further relates to methods and compositions for administering an agent which inhibits lymphedema or that induces lymphangiogenesis, such as VEGF-C, or a vector expressing VEGF-C. The administration can be sequential, simultaneous, or separated by a desired time period and can be by any suitable means.

Accordingly, the present invention relates to protein delivery, including by in vivo expression methods, to prevent or treat lymphedema. The present invention relates to such protein delivery to induce lymphangiogenesis. The present invention relates to such protein delivery for anti-lymphedema, e.g., to enhance development of lymph channels.

Various documents are cited in the following text, or in a reference section preceding the claims. Each of the documents cited herein, and each of the documents or references cited in each of those various documents, is hereby incorporated herein by reference. None of the documents cited in the following text is admitted to be prior art with respect to the present invention.

Reference is also made to U.S. patent application Serial No. 60/115,977, filed Jan. 15, 1999, especially to the extent that there are common inventors with this application, and that any compositions, methods, vectors, delivery systems and the like therein may be employed in the herein invention.

### BACKGROUND OF THE INVENTION

Lymphedema involving either the legs or arms is caused by obstruction of the lymphatic channels and is a common problem affecting tens of thousands of individuals throughout the world.

The most common etiology of lymphatic obstruction derives from several types of therapeutic interventions. For example, during the course of treatment of cancers, extensive dissection in regions containing the lymphatic channels that drain a limb can seriously compromise lymphatic drainage and ultimately lead to lymphedema. In particular, axillary dissections can lead to arm lymphedema and groin dissections can lead to leg lymphedema.

Often in the course of cancer therapy, multiple lymph nodes are removed to test for the presence of cancerous cells. This diagnostic procedure further compromises lymphatic drainage.

In addition, it often is deemed essential to radiate lymph nodes to eliminate remaining cancerous cells. Radiation seriously compromises the capacity of the lymphatic channels to drain the relevant limb, often resulting in lymphedema.

Lymphedema can also occur when non-cancer surgery is performed in a region in which lymphatics converge as they drain their relevant limb. A compromise in lymphatic drainage can occur during surgery of the shoulder or of the groin.

Also, lymphatic obstruction can occur spontaneously with no known precipitating cause and lead to lymphedema of the effected limb or limbs.

It appears that literature and patents have not heretofore addressed the problem of lymphedema as herein.

For instance, Witzenbichler et al., Am J Pathol 153(2):381-94 (1998), relates to vascular endothelial growth factor C (VEGF-C/VEGF-2) promoting angiogenesis in the setting of tissue ischemia. Cao et al., PNAS USA 95(24): 14389-94 (1998), pertains to vascular endothelial growth factor C inducing angiogenesis in vivo.

U.S. Pat. No. 5,919,459 concerns compositions and methods for treating cancer. U.S. Pat. Nos. 5,914,268 and 5,874,301 are directed to embryonic cell populations. U.S. Pat. No. 5,891,468 provides fusogenic liposome compositions.

U.S. Pat. No. 5,877,289 involves tissue factor compositions for the coagulation of vasculature. U.S. Pat. Nos. 5,863,538, 5,776,427, and 5,855,866 concern compositions and methods for targeting the vasculature of solid tumors and to methods for treating the vasculature of solid tumors, respectively.

U.S. Pat. No. 5,855,610 pertains to engineering of strong, pliable tissue. U.S. Pat. No. 5,776,755 is directed to FLT4, a receptor tyrosine kinase. U.S. Pat. No. 5,700,822 provides treatment of platelet derived growth factor related disorders such as cancer. U.S. Pat. No. 5,660,827 relates to antibodies that bind to endoglin.

Oh et al., Dev Biol 188(1):96-109 (1997), concerns VEGF and VEGF-C specific induction of angiogenesis and lymphangiogenesis in the differentiated avian cell chorioallantoic membrane. Kukk et al., Development 122(12):3829-37 (1996), involves VEGF-C receptor binding and pattern of expression with VEGFR-3 suggesting a role in lymphatic vascular development. Stavri et al., Circulation 92(1): 11-4 (1995), pertains to basic fibroblast growth factor upregulating the expression of vascular endothelial growth factor in vascular smooth muscle cells.

Oh et al., J Biol Chem 274(22):15732-9 (1999), is directed to hypoxia and vascular endothelial growth factor selectively up-regulating angiopoietin-2 in bovine microvascular endothelial cells. Lymboussake et al., Am J Pathol 153(2):395-403, involves expression of the vascular endothelial growth factor C receptor VEGFR-3 in lymphatic endothelium of the skin and in vascular tumors. Ristimaki et al., J Biol Chem 273(14):8413-8 (1998), relates to proinflammatory cytokines regulating expression of the lymphatic endothelial mitogen vascular endothelial growth factor-C. Jeltsch et al., Science 276(5317):1423-5 (1997), concerns hyperplasia of lymphatic vessels in VEGF-C transgenic mice (see also Science 277(5325):463 (1997)). And, Enholm et al., Oncogene 14(20):2475-83 (1997), is directed to a comparison of VEGF, VEGF-B, VEGF-C and Ang-1 mRNA regulation by serum, growth factors, oncoproteins and hypoxia.

Accordingly, it is believed that heretofore the application of proteins or vectors expressing proteins that enhance the development of lymphatic channels to individuals with obstructed lymph channels (lymphedema) has not been taught or suggested.

### OBJECTS AND SUMMARY OF THE INVENTION

It is therefore an object of the invention to provide methods and compositions for the prophylaxis of and/or therapy for lymphedema.

It is yet a further object of the invention to provide such methods and compositions for prophylaxis and/or therapy which comprise VEGF-C or an agent that enhances the development of lymphatic channels, e.g., lymphangiogenesis.

It is a still further object of the invention to provide such methods and compositions from in vitro and/or in vivo expression from plasmid DNA, or a vector system, such as a recombinant viral and/or DNA expression system; or from isolation from other sources, or from the administration of the protein itself.

It is a yet further object of the invention to provide such methods and compositions in conjunction with additional treatment methods and compositions; e.g., additional treatment methods and compositions for lymphedema or an underlying cause thereof, such as a cancer treatment.

The present invention thus provides methods and compositions for the prophylaxis of and/or therapy for lymphedema.

The present invention further provides such methods and compositions for prophylaxis and/or therapy which comprise an agent that enhances the development of lymphatic channels or lymphangiogenesis, such as VEGF-C, and/or that which stimulates VEGF-C expression and/or that which stimulates its interaction with its receptor (VEGF-C is a specific ligand for the VEGF receptor-3, or VEGFR-3, which is a specific marker for lymphatic endothelial cells) and/or an isoform of VEGF-C and/or a molecule having structural or functional homology to VEGF-C and/or a molecule that stimulates expression of VEGF-C and/or a molecule that participates in the signal transduction pathway of VEGF-C and/or in the signal transduction pathway of other molecules that stimulate the development of lymphatic channels or lymphangiogenesis and/or a molecule that activates other pathways to so stimulate the development of lymphatic channels or lymphangiogenesis and/or a portion or fragment of such a molecule that is active in inducing lymphangiogenesis and/or a vector that comprises a nucleic acid molecule or molecules encoding any one, or any combination, or all, of the foregoing.

Furthermore, with respect to sequences, nucleic acid molecules or sequences useful for expressing VEGF-C, and/or that which stimulates VEGF-C expression and/or that which stimulates its interaction with its receptor and/or an isoform of VEGF-C and/or a molecule having structural or functional homology to VEGF-C and/or a molecule that stimulates expression of VEGF-C and/or a molecule that participates in the signal transduction pathway of VEGF-C and/or in the signal transduction pathway of other molecules that stimulate development of lymphatic channels or lymphangiogenesis and/or other molecules that activate other pathways to so stimulate the development of lymphatic channels or lymphangiogenesis and/or an active fragment or portion thereof, they can include nucleic acid sequences that are capable of hybridizing under high stringency conditions or those having a high homology with nucleic acid molecules encoding VEGF-C, and/or that which stimulates VEGF-C expression and/or that which stimulates its interaction with its receptor and/or an isoform of VEGF-C and/or a molecule having structural or functional homology to VEGF-C and/or a molecule that stimulates expression of VEGF-C and/or a molecule that participates in the signal transduction pathway of VEGF-C and/or an active portion of such a molecule and/or a molecule otherwise employed in the invention (e.g., nucleic acid molecules in documents mentioned herein); and, “hybridizing under high stringency conditions” can be synonymous with “stringent hybridization conditions”, a term which is well known in the art; see, for example, Sambrook, “Molecular Cloning, A Laboratory Manual” second ed., CSH Press, Cold Spring Harbor, 1989; “Nucleic Acid Hybridisation, A Practical Approach”, Hames and Higgins eds., IRL Press, Oxford, 1985; both incorporated herein by reference.

With respect to nucleic acid molecules and polypeptides which can be used in the practice of the invention (e.g., the nucleic acid molecules encoding VEGF-C, and/or that which stimulates VEGF-C expression and/or that which stimulates its interaction with its receptor and/or an isoform of VEGF-C and/or a molecule having structural or functional homology to VEGF-C and/or a molecule that stimulates expression of VEGF-C and/or a molecule that participates in the signal transduction pathway of VEGF-C and/or in the signal transduction pathway of other molecules that stimulate the development of lymphatic channels or lymphangiogenesis and/or other molecules that activate other pathways to so stimulate the development of lymphatic channels or lymphangiogenesis and/or an active portion or fragment thereof; and VEGF-C, and/or that which stimulates VEGF-C expression and/or that which stimulates its interaction with its receptor and/or an isoform of VEGF-C and/or a molecule having structural or functional homology to VEGF-C and/or a molecule that stimulates expression of VEGF-C and/or a molecule that participates in the signal transduction pathway of VEGF-C and/or a molecule that in the signal transduction pathway of other molecules causing lymphangiogenesis or lymphatic channel development and/or other molecules that activate other pathways to so stimulate the development of lymphatic channels or lymphangiogenesis and/or an active portion or fragment of such a molecule, as polypeptides), these molecules advantageously have at least about 75% or greater homology or identity, advantageously 80% or greater homology or identity, more advantageously 85% or greater homology or identity, such as at least about 85% or about 86% or about 87% or about 88% or about 89% homology or identity, for instance at least about 90% or homology or identity or greater, such as at least about 91%, or about 92%, or about 93%, or about 94% identity or homology, more advantageously at least about 95% to 99% homology or identity or greater, such as at least about 95% homology or identity or greater e.g., at least about 96%, or about 97%, or about 98%, or about 99%, or even about 100% identity or homology, or from about 75%, advantageously from about 85% to about 100% or from about 90% to about 99% or about 100% or from about 95% to about 99% or about 100% identity or homology, with respect to the DNA sequences that encode VEGF-C, and/or that which stimulates VEGF-C expression and/or that which stimulates its interaction with its receptor and/or an isoform of VEGF-C and/or a molecule having structural or functional homology to VEGF-C and/or a molecule that stimulates expression of VEGF-C and/or a molecule that participates in the signal transduction pathway of VEGF-C and/or in the signal transduction pathway of other molecules causing lymphangiogenesis or lymphatic channel development and/or that activates other pathways to so stimulate the development of lymphatic channels or lymphangiogenesis and/or an active fragment or portion thereof, as well as with respect to the amino acid sequences for VEGF-C, and/or that which stimulates VEGF-C expression and/or that which stimulates its interaction with its receptor and/or an isoform of VEGF-C and/or a molecule having structural or functional homology to VEGF-C and/or a molecule that stimulates expression of VEGF-C and/or a molecule that participates in the signal transduction pathway of VEGF-C and/or a molecule that participates in the signal transduction pathway of VEGF-C and/or in the signal transduction pathway of other molecules causing lymphangiogenesis or lymphatic channel development and/or that activates other pathways to so stimulate the development of lymphatic channels or lymphangiogenesis and/or an active fragment or portion thereof, e.g., as set forth in herein cited documents (including subsequences thereof); and thus, the invention comprehends a vector encoding VEGF-C, and/or that which stimulates VEGF-C expression and/or that which stimulates its interaction with its receptor and/or an isoform of VEGF-C and/or a molecule having homology to VEGF-C and/or a molecule that stimulates expression of VEGF-C and/or a molecule that participates in the signal transduction pathway of VEGF-C, and/or a molecule that participates in the signal transduction pathway of VEGF-C and/or in the signal transduction pathway of other molecules causing lymphangiogenesis or lymphatic channel development and/or that activates other pathways to so stimulate the development of lymphatic channels or lymphangiogenesis and/or an active portion of such a molecule.

Nucleotide sequence homology can be determined using the “Align” program of Myers and Miller, (“Optimal Alignments in Linear Space”, CABIOS 4, 11-17, 1988, incorporated herein by reference) and available at NCBI. Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence similarity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur and Lipman, 1983 PNAS USA 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. RNA sequences within the scope of the invention can be derived from DNA sequences, by thymidine (T) in the DNA sequence being considered equal to uracil (U) in RNA sequences.

Additionally or alternatively, amino acid sequence similarity or identity or homology can be determined using the BlastP program (Altschul et al., Nucl. Acids Res. 25, 3389-3402, incorporated herein by reference) and available at NCBI. The following references (each incorporated herein by reference) also provide algorithms for comparing the relative identity or homology of amino acid residues of two proteins, and additionally or alternatively with respect to the foregoing, the teachings in these references can be used for determining percent homology or identity: Needleman S B and Wunsch C D, “A general method applicable to the search for similarities in the amino acid sequences of two proteins,” J. Mol. Biol. 48:444-453 (1970); Smith T F and Waterman M S, “Comparison of Bio-sequences,” Advances in Applied Mathematics 2:482-489 (1981); Smith T F, Waterman M S and Sadler J R, “Statistical characterization of nucleic acid sequence functional domains,” Nucleic Acids Res., 11:2205-2220 (1983); Feng D F and Dolittle R E, “Progressive sequence alignment as a prerequisite to correct phylogenetic trees,” J. of Molec. Evol, 25:351-360 (1987); Higgins D G and Sharp P M, “Fast and sensitive multiple sequence alignment on a microcomputer,” CABIOS, 5:151-153 (1989); Thompson J D, Higgins D G and Gibson T J. “ClusterW: improving the sensitivity of progressive multiple sequence alignment through sequence weighing, positions-specific gap penalties and weight matrix choice, Nucleic Acid Res., 22:4673-480 (1994); and, Devereux J, Haeberlie P and Smithies O, “A comprehensive set of sequence analysis program for the VAX,” Nucl. Acids Res., 12: 387-395 (1984).

Homology can also refer to a similar function, even in the complete absence of structural homology. Thus, the invention comprehends the use of any molecule that promotes lymphangiogenesis, even if it bears no sequence homology to VEGF-C.

Furthermore, as to nucleic acid molecules used in this invention (e.g., as in herein cited documents), the invention comprehends the use of codon equivalent nucleic acid molecules. For instance, if the invention comprehends “X” protein (e.g., Cp7 and/or Cp23 and/or Cp15/60) having amino acid sequence “A” and encoded by nucleic acid molecule “N”, the invention comprehends nucleic acid molecules that also encode protein X via one or more different codons than in nucleic acid molecule N.

Thus, the invention is not limited to VEGF-C, and it emcompasses that which stimulates VEGF-C expression and/or that which stimulates its interaction with its receptor and/or isoforms of VEGF-C and/or molecules homologous to VEGF-C, and/or molecules that stimulate expression of VEGF-C and/or molecules that participate in the signal transduction pathway of VEGF-C and/or a molecule that participates in the signal transduction pathway of VEGF-C and/or in the signal transduction pathway of other molecules causing lymphangiogenesis or lymphatic channel development and/or that activates other pathways to so stimulate the development of lymphatic channels or lymphangiogenesis and/or an active fragment or portion of any of the foregoing; and, nucleic acid molecules encoding any or all of the foregoing, and nucleic acid molecules having homology with nucleic acid molecules encoding any or all of the foregoing; and molecules that promotes lymphangiogenesis even in the absence of sequence homology to VEGF-C.

The present invention still further provides such methods and compositions from in vitro and/or in vivo expression from plasmid DNA, or a vector system, such as a recombinant viral and/or DNA expression system; or from isolation from other sources, or from the administration of the protein itself.

The administration can be after cancer treatment or in conjunction with it (e.g., at points during chemotherapy, radiation therapy, and the like, or after there has been surgery or removal of lymph nodes) or after or in conjunction with non-cancer surgery performed in a region in which lymphatics converge as they drain their relevant limb. Thus, the invention provides a therapeutic method for use with treatment of cancer or in conjunction with non-cancer surgery performed in a region in which lymphatics converge as they drain their relevant limb.

Similarly, the compositions of the invention can be administered before, during, or after any type of cancer treatment or therapy procedure (especially dissections, therapy such as radiation and/or chemotherapy, lymph node removal, etc.), or non-cancer surgery performed in a region in which lymphatics converge as they drain their relevant limb; e.g., before, to prevent, i.e., as a prophylaxis against, lymphedema; and during and after to prevent and/or control and/or treat lymphedema, for instance to prevent the development or progression of lymphedema.

Recombinant viral vectors, such as replication incompetent adenovirus, expressing the agent that induces development of lymphatic channels or lymphangiogenesis, e.g., VEGF-C and/or that which stimulates VEGF-C expression and/or that which stimulates VEGF-C expression or that which stimulates its interaction with its receptor or that which stimulates along any point of or any molecules involved in its signal transduction pathway can be administered in an amount of about 10⁷ pfu; thus, the inventive compositions can contain, and the inventive methods involve, administering a composition containing recombinant(s), at least this amount; more preferably about 10⁴ pfu to about 10¹⁰ pfu, e.g., about 10⁵ pfu to about 10⁹ pfu, for instance about 10⁶ pfu to about 10⁸ pfu. And, if more than one gene product is expressed by more than one recombinant, each recombinant can be administered in these amounts; or, each recombinant can be administered such that there is, in combination, a sum of recombinants comprising these amounts.

In naked DNA and DNA plasmid compositions, the dosage should be a sufficient amount of naked DNA or DNA plasmid to elicit a response analogous to compositions containing the agent that induces development of lymphatic channels or lymphangiogenesis, e.g., VEGF-C and/or that which stimulates VEGF-C expression and/or that which stimulates VEGF-C expression or that which stimulates its interaction or that which stimulates along any point of or any molecules involved in its signal transduction pathway; or to have expression analogous to dosages in such compositions; or to have expression analogous to expression obtained in vivo by other, e.g., viral, recombinant compositions. For instance, suitable quantities of naked DNA or plasmid DNA in naked DNA or DNA plasmid compositions can be 1 ug to 100 mg, preferably 0.1 to 10 mg, e.g., 500 ug, but lower levels such as 0.1 to 2 mg or even 1-10 ug, may be employed.

And, if more than one gene product is expressed by more than one recombinant and/or DNA (naked or plasmid) system, each recombinant and/or DNA system can be administered in these amounts; or, each recombinant and/or DNA system can be administered such that there is, in combination, a sum of recombinants and/or DNA comprising these amounts.

In protein form, the dosage should be a sufficient amount of the agent that induces development of lymphatic channels or lymphangiogenesis, e.g., VEGF-C and/or that which stimulates VEGF-C expression and/or that which stimulates VEGF-C expression or that which stimulates its interaction or activates other pathways to so stimulate the development of lymphatic channels or lymphangiogenesis or that which stimulates along any point of or any molecules involved in the signal transduction pathway of VEGF-C or in the signal transduction pathway of other molecules causing lymphatic channel development or lymphangiogenesis; for instance, suitable quantities of protein can be 1 ug to 100 mg, preferably 0.1 to 10 mg, e.g., 500 ug, but lower levels such as 0.1 to 2 mg or even 1-10 ug, may be employed.

And, if more than one protein is administered, each protein can be administered in these amounts; or, each protein can be administered such that there is, in combination, a sum of proteins comprising these amounts.

Subcutaneous, intradermal or intramuscular administration are presently preferred. Direct administration to the region, e.g., intraarterially or by direct injection, identified as containing deficient or obstructed lymphatic channels, is also possible. The protein can be administered in a suitable carrier or diluent; and, it can be in the form of liposomes or other carriers designed to efficiently deliver a protein or prolong protein half-life.

The invention further comprehends methods for preparing the compositions of the invention, as well as kits for compositions and methods of the invention. For instance, the invention comprehends a kit comprising an agent that induces development of lymphatic channels or lymphangiogenesis, e.g., VEGF-C and/or that which stimulates VEGF-C expression and/or that which stimulates VEGF-C expression or that which stimulates VEGF-C interaction or that which stimulates along any point of or any molecules involved in the signal transduction

pathway of VEGF-C or in the signal transduction pathway of other molecules causing lymphatic channel development or lymphangiogenesis or activates other pathways to so stimulate the development of lymphatic channels or lymphangiogenesis; the agents, if there is two or more, and/or the agent(s) and any carrier or diluent can be in separate containers; the agent(s)/carrier/diluent can be in separate containers contained in a package; and, the kit can optionally include instructions for the storage and/or use and/or admixture and/or administration of the agent(s)/carrier/diluent.

The term “comprising” can have the meaning ascribed to it in U.S. Patent Law; e.g., it can mean “including”.

These and other embodiments are disclosed or are obvious from and encompassed by, the following Detailed Description.

### DETAILED DESCRIPTION

Angiogenesis, or the development of new blood vessels, is a therapeutic target that has gained considerable interest over the past several years to treat ischemic syndromes of the heart and of the leg. One of the primary strategies for growing new blood vessels is the use of growth factors that stimulate the proliferation, migration, and tube formation of vascular endothelial cells. These strategies employ such angiogenic factors as vascular endothelial growth factor 165 (VEGF₁₆₅) and its various isoforms (such as VEGF₁₂₁) and homologous molecules such as VEGF-2, the FGF family of proteins, and several additional angiogenic factors.

In the course of these investigations it was found that endothelial cells lining lymphatic channels can also be stimulated by some of these factors. For example, VEGF receptor-3 (VEGFR-3) has been shown to be a specific marker for lymphatic endothelial cells in the human skin, and that VEGF-C (also called VEGF-2) is a specific ligand for this receptor (1). However, VEGFR-3 was also shown to be expressed in human saphenous vein and internal mammary artery (2).

The activity of VEGF-C is regulated by proinflammatory cytokines. Thus, IL-1B causes a concentration-dependant (increase in VEGF-C mRNA. TNFoc and IL-oc also elevate VEGF-C mRNA steady-state levels. Of note, hypoxia, which is an important inducer of VEGF₁₆₅ and VEGF₁₂₁ expression, has no effect on VEGF-C mRNA levels (3). These results demonstrate the primarily angiogenic protein VEGF₁₆₅ is regulated differently than the potentially lymphangiogenic protein VEGF-C. The data also suggest that proinflammatory cytokines regulate lymphatic vessel development via upregulation of VEGF-C.

Oh and colleagues studied the lymphatics of an avian chorioallantoic membrane (CAM) preparation. The chorioallantoic membrane is drained by lymphatic vessels accompanying arterioles, arteries, and veins. The investigators found that VEGF is angiogenic but not lymphangiogenic, whereas VEGF-C is lymphangiogenic, possessing strong chemoattractive activity for lymphatic endothelial cells, as well as having the capacity to induce proliferation of lymphatics endothelial cells and the development of new lymphatic sinuses (4).

Further confirmation of the lymphangiogenic potential of VEGF-C was determined in a transgenic mouse model. VEGF-C over-expression caused lymphatic, but not vascular, endothelial cell proliferation and vessel enlargement. The authors concluded that VEGF-C induces selective hyperplasia of the lymphatic vasculature (5).

Murine VEGF-C was cloned by Kukk et al (6). Murine VEGF-C is a dimer 85% homologous with the human VEGF-C amino acid sequence. Using the cloned murine gene, they found that VEGF-C mRNA was expressed in mesenchymal cells in regions where lymphatic vessels undergo sprouting, and in developing mesenterium, which is rich in lymphatic vessels.

Prophylactic/Therapeutic & Enhancing Strategy: The strategy employed by this invention is based on the concept that specific lymphangiogenesis interventions will cause the development of functioning lymphatic channels that can supplement the impaired functioning of lymphatic beds that have been decreased either by spontaneous disease or iatrogenically. The approach has the benefit of reducing the lymphedema of limbs supplied by these impaired lymphatic channels.

This invention is designed to employ gene therapy or protein delivery to prevent or treat lymphedema by enhancing the development of lymph channels or by stimulating lymphangiogenesis. The invention uses various strategies to suppress lymphedema such as the administration of an agent or a vector expressing an agent that induces development of lymphatic channels or lymphangiogenesis, e.g., VEGF-C and/or that which stimulates VEGF-C expression and/or that which stimulates VEGF-C expression or that which stimulates its interaction or that which, stimulates other pathways to so stimulate the development of lymphatic channels or lymphangiogenesis or that which stimulates along any point of or any molecules involved in the signal transduction pathway leading to lymphangiogenesis or lymphatic channel development. The agent could be in the form of a protein, or of a gene which expresses the protein. The gene could be delivered to the patient in a plasmid, or in any other vector, including a viral vector. Delivery to patient will vary depending on the clinical situation; but, the time and amount and route or method of delivery can be determined by this disclosure and the knowledge in the art, without undue experimentation.

Thus, the present invention includes compositions and methods for preventing or treating lymphedema. The present invention includes compositions comprising an agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymphangiogenesis or that which stimulates along any point of or any molecules involved in the signal transduction pathway leading to lymphangiogenesis or lymphatic channel development; as well as methods comprising the administration of such agent(s), e.g., individually, or separately, or sequentially or the like or in conjunction with other treatment or therapy such as cancer treatment or therapy or therapy or treatment involving non-cancer surgery in a region which lymphatics converge as they drain their relevant limb. Any or all of these agents can be present in the composition by way of a vector which expresses the agent in vivo.

As to cloning and expression VEGF-C or VEGF-2, it is noted that Kukk et al. (6) cloned munne VEGF-C (see also Parast et al. Biochemistry 37(47):16788-801 (November 1998) (VEGFR2 TK catalytic domain has been cloned and expressed via a baculovirus expression system); Pepper et al. J Cell Physiol 177(3):439-52 (December 1998) (VEGF-C has a potent synergistic effect on the induction of angiogenesis and VEGE, bFGF and VEGF-C are capable of altering endothelial cell extracellular proteolytic activity)).

As to administration of an agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymphangiogenesis or that which stimulates along any point of or any molecules involved in the signal transduction pathway leading to lymphangiogenesis or lymph channel development, these agent(s) can be administered by any suitable means, and such means can include the proteins, naked plasmid DNA, viral vectors, an intra-arterial infusion, direct injection, and the like (See Witzenbichler et a!. Am J Pathol 153(2):381-94 (August 1998): VEGF-C promotes angiogenesis; demonstrates administration of VEGF-C by means of naked plasmid DNA (pcVEGF-C 500 microg), polymer coating of an angioplasty balloon (n=8) or as a recombinant human protein (rhVEGF-C 500 microg) by direct intra-arterial infusion; WO 98/33510 (vectors including viral vectors, plasmid vectors)).

An agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymphangiogenesis or that which stimulates along any point of or any molecules involved in the signal transduction pathway leading to lymphangiogenesis or lymph channel development, can be obtained by purification from natural sources or from purification from recombinant sources; and, techniques for such purifications or for protein purification are generally known and require no undue experimentation by the skilled artisan.

The methods for making and/or administering a vector or recombinant for expression of such agents either in viva or in vitro can be by or analogous to the methods disclosed in: U.S. Pat. Nos. 4,603,112, 4,769,330, 5,174,993, 5,505,941, 5,338,683, 5,494,807, 4,722,848, WO 94/16716, WO 96/39491, Paoletti, “Applications of pox virus vectors to vaccination: An update,” PNAS USA 93:11349-11353, October 1996, Moss, “Genetically engineered poxviruses for recombinant gene expression, vaccination, and safety,” PNAS USA 93:11341-11348, October 1996, Smith et al., U.S. Pat. No. 4,745,051 (recombinant baculovirus), Richardson, C. D. (Editor), Methods in Molecular Biology 39, “Baculovirus Expression Protocols” (1995 Humana Press Inc.), Smith et al., “Production of Huma Beta Interferon in Insect Cells Infected with a Baculovirus Expression Vector,” Molecular and Cellular Biology, Dec., 1983, Vol. 3, No. 12, p. 2156-2165; Pennock et al., “Strong and Regulated Expression of Escherichia coli BGalactosidase in Infect Cells with a Baculovirus vector,” Molecular and Cellular Biology March 1984, Vol. 4, No. 3, p. 399-406; EPA 0 370 573, U.S. application Ser. No. 920,197, filed Oct. 16, 1986, EP Patent publication No. 265785, U.S. Pat. No. 4,769,331 (recombinant herpesvirus), Roizmian, “The function of herpes simplex virus genes: A primer for genetic engineering of novel vectors,” PNAS USA 93:11307-11312, October 1996, Andreanasky et al., “The application of genetically engineered herpes simplex viruses to the treatment of experimental brain tumors,” PNAS USA 93:11313-11318, October 1996, Robertson et al. “Epstein-Barr virus vectors for gene delivery to B lymphocytes,” PNAS USA 93:11334-11340, October 1996, Frolov et al., “Alphavirus-based expression vectors: Strategies and applications,” PNAS USA 93:11371-11377, October 1996, Kitson et al., J. Virol. 65, 3068-3075, 1991; U.S. Pat. Nos. 5,591,439, 5,552,143 (recombinant adenovirus), Grunhaus et al., 1992, “Adenovirus as cloning vectors,” Seminars in Virology (Vol. 3) p. 23 7-52, 1993, Ballay et al. EMBO Journal, vol. 4, p. 386 1-65, Graham, Tibtech 8, 85-87, April, 1990, Prevec et al., J. Gen Virol. 70, 429-434, PCT WO92/11525, Felgner et al. (1994), J. Biol. Chem. 269, 2550-2561, Science, 259:1745-49, 1993 and McClements et al., “Immunization with DNA vaccines encoding glycoprotein D or glycoprotein B, alone or in combination, induces protective immunity in animal models of herpes simplex virus-2 disease,” PNAS USA 93:11414-11420, October 1996, and U.S. Pat. Nos. 5,591,639, 5,589,466, and 5,580,859 relating to DNA expression vectors, inter alto. See also WO 98/33510; Ju et al., Diabetologia, 41:736-739, 1998 (lentiviral expression system); Sanford et al., U.S. Pat. No. 4,945,050 (method for transporting substances into living cells and tissues and apparatus therefor); Fischbach et al. (Intracel), WO 90/01543 (method for the genetic expression of heterologous proteins by cells transfected); Robinson et al., seminars in IMMUNOLOGY, vol. 9, pp. 271-283 (1997) (DNA vaccines); Szoka et al, U.S. Pat. No. 4,394,448 (method of inserting DNA into living cells); and McCormick et al., U.S. Pat. No. 5,677,178 (use of cytopathic viruses for therapy and prophylaxis of neoplasia).

The expression product generated by vectors or recombinants in this invention can also be isolated from infected or transfected cells and used to prepare compositions for administration to patients.

More generally, compositions for use in the invention can be prepared in accordance with standard techniques well known to those skilled in the pharmaceutical or medical arts. Such compositions can be administered in dosages and by techniques well known to those skilled in the medical ads taking into consideration such factors as the age, sex, weight, and condition of the particular patient, and the route of administration. The compositions can be administered alone, or can be co-administered or sequentially administered with other compositions of the invention or with other prophylactic or therapeutic compositions.

Examples of compositions of the invention include liquid preparations for orifice, e.g., oral, nasal, anal, genital (e.g., vaginal), vascular and/or SMC, etc., administration such as suspensions, syrups or elixirs; and, preparations for parenteral, subcutaneous, intradermal, intramuscular, intravenous, intraarterial (e.g., at site region deficient in or having obstructed lymphatic channels), intralymphatic, or intraperitoneal administration (e.g., injectable administration) such as sterile suspensions or emulsions. In such compositions the active agent be in admixture with a suitable carrier, diluent, or excipient such as sterile water, physiological saline, glucose or the like. The vector or protein can be in the form of liposomes or other carriers designed to more efficiently deliver the protein or vector or to prolong its half-life.

The compositions of the invention may be packaged in a single dosage form for immunization by parenteral (i.e., intramuscular, intradermal or subcutaneous) administration or orifice administration, e.g., perlingual (i.e., oral), intragastric, mucosal including intraoral, intraanal, intravaginal, intravenous, intralymphatic, intraarterial (e.g., at site of deficiency or obstruction in lymphatic channels), intraperitoneal, and the like administration. Accordingly, compositions in forms for such administration routes are envisioned by the invention. And again, the effective dosage and route of administration are determined by known factors, such as age, sex, weight, condition and nature of patient, as well as LD₅₀ and other screening procedures which are known and do not require undue experimentation.

Dosages of each active agent can range from a few to a few hundred micrograms, e.g., 5 to 500 mg. An inventive vector or recombinant expressing an agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymphangiogenesis or that which stimulates along any point of or any molecules involved in the signal transduction pathway leading to lymphangiogenesis or lymph channel development, can be administered in any suitable amount to achieve expression at these dosage levels. The inventive vector or recombinant can be administered to a patient or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu. And, if more than one gene product is expressed by more than one recombinant, each recombinant can be administered in these amounts; or, each recombinant can be administered such that there is, in combination, a sum of recombinants comprising these amounts. Other suitable carriers or diluents can be water or a buffered saline, with or without a preservative. The expression product or isolated product or vector or recombinant may be lyophilized for resuspension at the time of administration or can be in solution.

In plasmid compositions, the dosage should be a sufficient amount of plasmid to elicit a response analogous to compositions wherein the agent or agents are directly present; or to have expression analogous to dosages in such compositions; or to have expression analogous to expression obtained in vivo by recombinant compositions. For instance, suitable quantities of plasmid DNA in plasmid compositions can be 1 ug to 100 mg, preferably 0.1 to 10 mg, e.g., 500 micrograms, but lower levels such as 0.1 to 2 mg or preferably 1-10 ug may be employed. Documents cited herein, for instance, regarding vector expression and/or DNA plasmid vectors may, be consulted for the skilled artisan to ascertain other suitable dosages for expression vector and/or DNA plasmid vector compositions of the invention, without undue experimentation.

For treatment of lymphedema, the compositions comprising an agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymphangiogenesis and/or that which stimulates any along any point of or any molecules involved in the signal transduction pathway leading to lymphangiogenesis or lymph channel development (and/or vector(s) expressing one or more of these agent(s)), alone or with other treatment (e.g., cancer treatment therapy and/or non-cancer surgery in a region in which lymphatics converge and/or treatment, therapy for such non-cancer surgery), may be administered as desired by the skilled medical practitioner, from this disclosure and knowledge in the art, e.g., at the first signs or symptoms of the condition for which the other treatment is being administered, or as soon thereafter as desired by the skilled medical practitioner, without any undue experimentation required; and, the administration of the compositions, alone or with other treatment, may be continued as a regimen, e.g., monthly, bimonthly, biannually, annually, or in some other regimen, by the skilled medical practitioner for such time as is necessary to prevent or treat lymphedema and/or the other condition for which treatment is being administered, without any undue experimentation required. For treatment of lymphedema, the compositions of this invention, alone or with other treatment, may be administered at the first signs or symptoms of lymphedema or a condition that can lead to it, or as soon thereafter as desired by the skilled medical practitioner, without any undue experimentation required; and, the administration of the compositions, alone or with other treatment, may be continued as a regimen, e.g., monthly, bimonthly, biannually, annually, or in some other regimen, by the skilled medical practitioner for such time as is necessary to prevent lymphedema or further symptoms or signs thereof, without any undue experimentation required.

For prevention of lymphedema, the compositions, alone or with other treatment, may be administered at the first indication of the patient being prone to lymphedema (e.g., detection, treatment of cancer or of administration of treatment/procedure that can induce lymphedema such as non-cancer surgery in region in which lymphatics converge), or as soon thereafter as desired by the skilled medical practitioner, e.g., within six months prior to, immediately prior to, or at or during treatment/procedure that can induce lymphedema (e.g., cancer treatment/therapy/procedure(s) and non-cancer treatment/therapy/procedure(s) such as those alluded to elsewhere herein), in any desired regimen such as a single administration or multiple administrations or in a regimen as desired, e.g., monthly, bimonthly, biannually, or within a year after, or annually or regularly or any combination thereof, for such time as is necessary to prevent lymphedema or symptoms or signs thereof, without any undue experimentation required.

The compositions of the invention can be administered before, during or immediately after a cancer treatment/therapy/procedure or non-cancer treatment/therapy/procedure prone to inducing lymphedema to induce maximal responses at that time, since process that may lead to lymphedema can happen quickly.

A better understanding of the present invention and of its many advantages will be had from the following examples, given by way of illustration.

### EXAMPLES

### Example 1

### Lymphedema Prevention/Treatment

Mice and/or pigs are subjected to lymphedema causing treatment/procedure/therapy, e.g., non-cancer surgery in region(s) in which lymphatics converge, as well as cancer treatment/therapy/procedure(s), e.g., radiation, lymph node removal, dissection in regions containing lymphatic channels, chemotherapy (with such mice or pigs either cancer-free or having tumor(s) such as tumor(s);commonly induced in such animals) Certain of those animals are given “treatment” (an agent that induces development of lymphatic channels or lymphangiogenesis, e.g., VEGF-C and/or that which stimulates VEGF-C expression and/or that which stimulates VEGF-C expression and/or that which stimulates its interaction or that which stimulates other pathways to so stimulate the development of lymphatic channels or lymphangiogenesis and/or that which stimulates along any point of or any molecules involved in the signal transduction pathway leading to lymphangiogenesis or lymph channel development (and/or vector(s) expressing one or more of these agent(s)).

Protocol: in a first set of such animals treatment is administered in the form of either a protein or a vector expressing a nucleic acid molecule or gene encoding the protein, during and after the lymphedema causing event; in a second set of such animals no treatment is administered prior to, during or after the lymphedema causing event. The treatment comprises an agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymnphangiogenesis or that which stimulates along any point of or any molecules involved in the signal transduction pathway leading to lymphangiogenesis or lymph channel development and/or vector(s) expressing one or more of these agent(s)) such as adenoviral vectors. The administration is intraarterially or by direct injection (e.g., the Infusate catheter (Interventional Technology)) in amounts as herein described (taking into consideration the weight or mass of the animal, especially in relation to the average weight or mass of a human).

Endpoint Measurements:

A) Tissue is obtained from each of 2 treated, and each of 2 untreated animals sacrificed 2 h, 6 h, 24 h, and 14 days after injury and analysed for one or more or any or all of:

VEGF-C protein (by immunohistochemistry and/or by Western analysis);

observation of lymphatic channels regarding amount thereof and possible blackage/obstruction or lack thereof

B) Lymphatics are injected with dye in each of 8 treated, and each of 8 untreated animals sacrificed at 28 days after injury and analyzed and observation of lymphatic channels regarding amount thereof and possible blockage/obstruction or lack thereof.

Volume of the normal and lymphatic-blocked limb are measured.

Results confirm that administration of a VEGF-C and/or vector expressing it can prevent, treat and/or control lymphedema.

### Example 2

### Formulations and Use

The agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymphangiogenesis or that which stimulates along any point of or any molecules involved in the signal transduction pathway leading to lymphangiogenesis or lymph channel development (and/or vector(s) expressing one or more of these agent(s)) are admixed with carrier, diluent etc. and optionally, prior thereto can be formulated into liposomes or other forms that enhance the half-life of the protein(s) and/or vector(s), as herein described in amounts as herein described to obtain formulations. DNA encoding an agent that induces development of lymphatic channels or lymphangiogenesis, e.g., VEGF-C and/or that which stimulates VEGF-C expression and/or that which stimulates VEGF-C expression or that which stimulates its interaction or that which stimulates other pathways to so stimulate the development of lymphatic channels or lymphangiogenesis or that which stimulates along any point of or any molecules involved in the signal transduction pathway leading to lymphangiogenesis or lymph channel development is/are used to generate recombiriants and DNA expression systems expressing these agents; and, these recombinants and DNA expression systems are admixed with carrier, diluent, etc., and optionally, prior thereto can be formulated into liposomes or other forms that enhance the half-life of the vector(s), as herein described to obtain formulations. Patients are administered the formulations as herein described for the prevention and/or treatment of lymphedema, including in a manner analogous to gene therapy directed against SMC proliferation, as described in literature or documents cited herein or in documents cited in literature or documents cited herein.

Having thus described in detail preferred embodiments of the present invention, it is to be understood that the invention defined by the appended claims is not to be limited by particular details set forth in the above description as many apparent variations thereof are possible without departing from the spirit or scope thereof.

I claim:

1. A method for treating lymphedema comprising administration to a patient afflicted with lymphedema an amount of a compound or expression system capable of stimulating the development of lymphatic channels or lymphangiogenesis effective to alleviate said lymphedema.

2. The method of claim 1 wherein said compound is VEGF-C or a compound that stimulates the expression of VEGF-C.

3. The method of claim 1 wherein said compound stimulates the interaction of VEGF-C with a receptor therefor.

4. The method of claim 1 wherein said compound is a fragment of VEGF-C capable of stimulating the development of lymphatic channels or lymphangiogenesis.

5. The method of claim 1 wherein said compound is a compound having structural or functional homology to VEGF-C and being capable of stimulating the development of lymphatic channels or lymphangiogenesis.

6. The method of claim 2 wherein said compound that stimulates the production of VEGF-C is a compound that participates in a signal transduction pathway controlling the expression of VEGF-C.

7. The method of claim 1 wherein said expression system causes the expression of VEGF-C, an active fragment of VEGF-C, a compound having structural or functional homology to VEGF-C and being capable of stimulating the development of lymphatic channels or lymphangiogenesis, a compound that stimulates the interaction of VEGF-C with a receptor therefor, or a compound that participates in a signal transduction pathway controlling the expression of VEGF-C.

8. The method of claim 7 wherein said expression system comprises an adenovirus, a poxvirus, a baculovirus or a DNA plasmid.

9. A composition for treatment of lymphedema comprising a compound selected from the group consisting of VEGF-C, an active fragment of VEGF-C, a compound having structural or functional homology to VEGF-C and being capable of stimulating the development of lymphatic channels or lymphangiogenesis, a compound that stimulates the interaction of VEGF-C with a receptor therefor, and a compound that participates in a signal transduction pathway controlling the expression of VEGF-C, said compound being dispersed in a pharmaceutically acceptable non-toxic vehicle.
